SEQUENCE LISTING (7 sequences)

SEQ ID NO: 1 - DNA, 1649 bp, Mus musculus
  CDS (36)..(1469)
ctagagcgtgctgacaggggtctcaagtgtggggcatggaccattcactgggatggcaagggaactctgtccccgaggacgggactgaagctgggatcaagcacttcctggaggacagcagtgatgatgctgagctgagcaagttcgtgaaggatttcccaggaagcgaaccctaccactcagcggagtccaagacaagggtggccaggccccagatcttggagccaaggccccagagcccagacctctgtgatgatgacgtggagtttagaggctccttgtggccccagccctctgacagtcagcagtacttcagtgccccagcccctctcagcccttcctccaggccccgcagtccatggggcaagcttgatccttatgattcctctgaggatgacaaggagtatgtgggctttgcaaccctccccaatcaagtccacaggaagtctgtgaagaaaggctttgactttacactcatggtggcaggagaatctggtctgggtaaatccactcttgtcaacagcctcttcctcactgacttgtaccgggatcggaaactgctgggcgccgaagagcggatcatgcaaaccgtggagattactaagcacgcagtggatatagaagagaagggagtgaggctgcggctcaccattgtggacactccaggatttggggatgcagtcaacaacacagagtgctggaagcctgtggccgaatacatcgaccagcagtttgagcagtacttccgagacgagagtggcctgaaccgcaagaacatccaggacaaccgggtgcactgctgcctgtacttcatctccccgtttggccacgggctccggccattggatgttgaattcatgaaggccctgcatcagcgggtcaacattgtgcctatcttggctaaggcggacacactgacgcctcctgaagtggaccgaaagaaatgcaaaatccgggaggagatcgagcactttggaatcaagatctatcagttcccagactgtgattccgatgaggacgaggacttcaaattacaggaccaagccctaaaggaaagcatcccatttgcggtgattggcagcaacactgtggtagaagccagggggcggagagttcgaggccgcctctacccttggggcatcgtggaagtggaaaacccaggtcactgcgactttgtcaagttgaggacgatgctggtgcgtacccacatgcaggacctaaaggatgtgacccgagagacacactacgagaactacagggcacagtgtatccagagcatgacccggctagtagtgaaggaacggaatcgcaacaaactgacaagagagagtggtactgacttccctatccctgctgtcccaccagggacagatccagaaactgagaagctaatccgggagaaagacgaagagctgcggcggatgcaggagatgttacacaaaatccaaagacagatgaaggagactcactaactggcttttggacctgaatatttaaatctcttcttcctgcctatgccagcctctattccctgcatcagctctgctcaggaccccccgcagctcctaccagttcgccttatatccctgctgacttctcaagagactcagaggaaataaactaacctatatgtggcaaaaaaaaaaaaa

SEQ ID NO: 2 - DNA, 134 bp, Mus musculus
agccaaggccccagagcccagacctctgtgatgatgacgtggagtttagaggctccttgtggccccagccctctgacagtcagcagtacttcagtgccccagcccctctcagcccttcctccaggccccgcagt

SEQ ID NO: 3 - DNA, 171 bp, Mus musculus
agacgagagtggcctgaaccgcaagaacatccaggacaaccgggtgcactgctgcctgtacttcatctccccgtttggccacgggctccggccattggatgttgaattcatgaaggccctgcatcagcgggtcaacattgtgcctatcttggctaaggcggacacactgac

SEQ ID NO: 4 - DNA, 191 bp, Mus musculus
aagggggaaggaaccaatcaggaagccagatgacagcaggagcatcaagagaagcctgtttctgtgtgaggacccttgcctggagataaagttacacctagagcgtgctgacaggggtctcaagtgtggggcatggaccattcactgggatggcaagggaactctgtccccgaggacgggactgaagctgg

SEQ ID NO: 5 - DNA, 2225 bp, Homo sapiens
  5′UTR (1)..(96)
  CDS (97)..(720)
  3′UTR (721)..(2225)
agcatcaaaacaaggctgtttctgtgtgtgaggaactttgcctgggagataaaattagacctagagctttctgacagggagtctgaagcgtgggacatggaccgttcactgggatggcaagggaattctgtccctgaggacaggactgaagctgggatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggtagagcctccagccctgcctttgcctttcagtgggctgctgcaggaagaccgggggcagggagcagagtgtgtgtgtgtgtgtgtgtgtgtgtgtgtgtgtgtgtgtttgtgtgtgtgtgtatctgggacctatttcagtcctgtgtcagccctagctccaagatatctgcccccaagggcactggaaatttgcagtttcagcaagggcaggaggcccagctggtggcctcagatgggaactcacagaagtctggcactgcttttttaaggctggggcaaaggcctgaaagggagagaagattggcgctgggtgccggggcccctttggctcctcaccgtgatgcattctgccttcctgtctactaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggctccggccattggatgttgaattcatgaaggccctgcatcagcgggtcaacatcgtgcctatcctggctaaggcagacacactgacacctcccgaagtggaccacaagaaacgcaaaatccgggaggagattgagcattttggaatcaagatctatcaattcccagactgtgactctgatgaggatgaggacttcaaattgcaggaccaagccctaaaggaaagcatcccatttgcagtaattggcagcaacactgtagtagaggccagagggcggcgagttcggggtcgactctacccctggggcatcgtggaagtggaaaacccagggcactgcgactttgtgaagctgaggacaatgctggtacgtacccacatgcaggacctgaaggatgtgacacgggagacacattatgagaactaccgggcacagtgcatccagagcatgacccgcctggtggtgaaggaacggaatcgcaagtatgaccagaagccaggacaaagctggcagggggagatcccaagcctagccttgggtgagaccaagccctacttttgttcttctataggccctgggctcaatctaagcgggtgctggggtcctcctcgccttatcaacccttttctccctttagcaaactgactcgggaaagtggtaccgacttccccatccctgctgtcccaccagggacagatccagaaactgagaagcttatccgagagaaagatgaggagctgcggcggatgcaggagatgctacacaaaatacaaaaacagatgaaggagaactattaactggctttcagccctggatatttaaatctcctcctcttcttcctgtccatgccggcccctcccagcaccagctctgctcaggccccttcagctactgccacttcgccttacatccctgctgactgcccagagactcagaggaaataaagtttaataaatctgtaggtgg

SEQ ID NO: 6 - protein, 478 aa, Mus musculus
MDHSLGWQGNSVPEDGTEAGIKHFLEDSSDDAELSKFVKDFPGSEPYHSAESKTRVARPQILEPRPQSPDLCDDDVEFRGSLWPQPSDSQQYFSAPAPLSPSSRPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGLRPLDVEFMKALHQRVNIVPILAKADTLTPPEVDRKKCKIREEIEHFGIKIYQFPDCDSDEDEDFKLQDQALKESIPFAVIGSNTVVEARGRRVRGRLYPWGIVEVENPGHCDFVKLRTMLVRTHMQDLKDVTRETHYENYRAQCIQSMTRLVVKERNRNKLTRESGTDFPIPAVPPGTDPETEKLIREKDEELRRMQEMLHKIQRQMKETH

SEQ ID NO: 7 - protein, 208 aa, Homo sapiens
MDRSLGWQGNSVPEDRTEAGIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEVEPPALPLPFSGLLQEDRGQGAECVCVCVCVCVCVCLCVCVSGTYFSPVSALAPRYLPPRALEICSFSKGRRPSWWPQMGTHRSLALLF

(19) United States
(12) Patent Application Publication
(10) Pub. No.: US 2006/0242723 A1
(43) Pub. Date: Oct. 26, 2006

(54) Chimeric mouse with regulated bradeion gene expression

(75) Inventors: Manami Tanaka, Ibaraki (JP); Tomoo Tanaka, Kanagawa (JP)

Correspondence Address: FOLEY AND LARDNER LLP;SUITE 500, 3000 K STREET NW, WASHINGTON, DC, 20007, US

(73) Assignee: National Institute of Advanced Industrial Science & Technology, Tokyo (JP)

(21) Appl. No.: 10/530,401

(22) PCT Filed: Oct. 11, 2002
(86) PCT No.: PCT/JP02/10599

§ 371 Date: Apr. 7, 2005

(51) Int. Cl. A01K 67/027; C12N 5/06

(52) U.S. Cl. 800/18; 435/354

(57) ABSTRACT

The present invention relates to a chimeric mouse having an endogenous Bradeion gene, the gene expression of which has been suppressed, and a cell derived from a mouse embryonic stem cell, which is collected from such chimeric mouse.

### TECHNICAL FIELD

The present invention relates to a chimeric mouse having an endogenous Bradeion gene, the gene expression of which has been suppressed by genetic alteration.

### BACKGROUND ART

A revolution in molecular medicine in the 21st century aims at construction of a controlled monitoring system tailored to each individual's properties on the basis of the genes and/or substances associated with diseases through post-genome projects. More specifically, on the basis of the concept of “Quality of Life,” such projects aim at detection of high risk groups for social life-threatening diseases, such as genetic diseases, cancers, or nerve degenerative diseases (diagnosis and gene monitoring); discovery of risk genes; and searches for sensitivity to treatments (for example, drugs for administration or gene therapy), so as to establish a medical treatment system compatible for the genetic type of each individual. Since many diseases, including cancers, are developed due to multi-gene effects and are also largely affected by environmental factors, it cannot be said with assurance that the control of certain factors would mean that a certain disease would never be developed. However, it is possible to control a developed disease, that is, to take measures to control a developed disease through the development of so-called control technology.

Based on this concept, recently, the development of the technology for controlling canceration and/or immortalization of cells is particularly actively being studied through analyses of cell division suppressors of the cerebral nervous system and the like. Through such studies, various molecular bases associated with cell growth, cell division, and/or canceration have been revealed. A human Bradeion protein is a cellular life-span-controlling factor associated with long-term survival of cerebral neurons after their generation and/or differentiation. As a result of such studies, patent applications for inventions relating to such a human Bradeion protein have already been submitted by the National Institute of Advanced Industrial Science and Technology (ex. the Agency of Industrial Science and Technology) (JP Patent Publication (Kokai) No. 2000-139470 A, JP Patent Publication (Kokai) No. 2001-161384 A, and U.S. patent application Ser. No. 09/440,936). These studies have clarified that the Bradeion protein is specifically expressed in adult cerebral nerve system cells, colon cancer and/or prostate cancer cells, and skin cancer. Hence, it has been revealed that the Bradeion is not only useful as a target for early diagnosis of cell mutations or the like, but it also meets various requirements as a specific inhibitor and as a target for gene therapy (see the above group of patents, and Tanaka, M. et al., Biochem. Biophys. Res. Commun. (2001) 286, 547-553).

### DISCLOSURE OF THE INVENTION

Even if a signaling substance that can be a target is discovered, analyses limited to the level of culture cells are insufficient to achieve the original purpose; that is, disease control or development of technology for controlling gene functions making use of such discovery. Further analyses using biological individual models are required. Hence, there is a need to establish an appropriate model animal. For this purpose, gene-altered animals including various knockout mice for specific genes are currently being produced and used.

However, in cases in which the expression of a specific gene is suppressed from the time of generation, it is currently difficult to substantially predict how the effect of such suppression affects morphogenesis and biological functions in the generation processes.

It is an object of the present invention to provide a novel knockout chimeric mouse, which is useful as a model animal and an animal for genetic breeding.

As a result of intensive studies directed towards solving the above-described problems, the present inventors have succeeded in producing a mouse embryonic stem cell having at least one endogenous Bradeion gene, where the gene expression of which has been suppressed by gene engineering techniques. The present inventors have produced chimeric mice by introducing the embryonic stem cells and clarified that such chimeric mice then exhibit hypoplasia in the overall cerebral nervous system and malformation such as generalized decreased growth, cranial dysplasia, and visual disorders. Thus the present inventors have completed the present invention.

The present invention is as follows.
(1) A chimeric mouse generated from a mouse embryo, wherein the mouse embryo has a mouse embryonic stem cell introduced therein that has a genomic DNA containing an endogenous Bradeion gene, the gene expression of which has been suppressed.

Such an endogenous Bradeion gene, the gene expression of which has been suppressed, can be a gene that has been genetically altered to encode a Bradeion protein having decreased biological activity or a Bradeion protein lacking biological activity. Furthermore, the endogenous Bradeion gene, the gene expression of which has been suppressed, can be a gene that has been genetically altered to lack the entire coding region.
(2) The chimeric mouse of (1) above, wherein the mouse embryonic stem cell is derived from a PJ1-5 line and the mouse embryo is derived from a C57BL/6 mouse.
(3) The chimeric mouse of (1) or (2) above, wherein the mouse embryo is selected from the group consisting of 8-cell-stage embryos, morulae, and blastocysts.
(4) The chimeric mouse of (1) to (3) above, which exhibits malformation.

Examples of such malformation include cranial dysplasia, visual disorders, and generalized decreased growth.
(5) The chimeric mouse of (1) to (4) above, wherein the chimeric rate is 90% or more and less than 98%.
(6) A cell derived from the mouse embryonic stem cell, which is collected from the chimeric mouse of (1) to (5) above.

The present invention will be described in detail below.

It has been reported that a Bradeion gene is associated with long-term survival of cerebral neurons. Furthermore, for example, the expression of the gene is recognized specifically in the adult brain and the like in case of humans, but the expression is not recognized in human fetuses. Hence, the functions of the Bradeion gene in generation processes have remained unknown. Regarding this point, it has been revealed for the first time in the present invention that chimeric mice, wherein the expression of the endogenous Bradeion gene has been suppressed, exhibit hypoplasia of the cerebral nervous system and malformation as described above. Based on this finding, it has been revealed that the chimeric mouse of the present invention can serve as an appropriate model animal for elucidating molecular mechanisms, by which the above hypoplasia in the cerebral nervous system and malformation are caused, and for developing methods for treating or controlling disorders and diseases associated with such hypoplasia in the cerebral nervous system and malformation.

Therefore, the present invention has been completed by discovering that a chimeric mouse, which is produced by introducing a mouse embryonic stem cell having at least one endogenous Bradeion gene, the gene expression of which has been suppressed from the time of generation, is useful as a model animal or an animal for genetic breeding.

The present invention relates to a chimeric mouse with suppressed Bradeion gene expression, which is produced by producing a mouse embryonic stem cell having a genomic DNA, wherein the expression of at least one endogenous Bradeion gene has been suppressed, and then introducing such embryonic stem cell into a mouse early embryo so as to cause the generation thereof. The chimeric mouse of the present invention is characterized in that it develops hypoplasia in the cerebral nervous system and various types of malformation as a result of suppressing the expression of the Bradeion gene from the time of generation.

1. Bradeion Gene

Bradeion is a protein that is known to exist specifically in the human adult brain or the like and is associated with the long-term survival of cerebral neurons. The protein has a structure similar to that of a substance (septin family) associated with control of cell division and growth. At the same time, the protein has a structure as a determinant for determining cellular life span (causing programmed cell death). From preliminary experiments or the like, the functions of Bradeion have been elucidated. It has been found that Bradeion is a cell-division-controlling factor belonging to the septin family, which exhibits specific expression in cancer cells, and that it plays a role as an MAP kinase signaling cascade or as a motor pump of cell growth equipment at the final point of cell division. In humans, the Bradeion protein has been known as two types of transcriptional and translational products encoded by a single Bradeion gene, that is, α type and β type. In addition, in humans, such tissue-specific expression of the Bradeion protein has been recognized also in colon cancer tissues and skin cancer tissues (Tanaka et al., Biochemical and Biophysical Research Communications 286, 547-553 (2001)). Furthermore in mice, the presence of a homologue of β type Bradeion protein has been reported (JP Patent Publication (Kokai) No. 2000-139470 A).

According to these findings, the present invention has been completed based on the idea that if a chimeric mouse having an endogenous Bradeion gene, the gene expression of which has been suppressed, can be produced, such a mouse may be useful as a model animal relating to disorders and/or diseases associated with cerebral neurons and relating to cell canceration.

Regarding a subject endogenous Bradeion gene, the gene expression of which is suppressed, either one of or both allelic genes of an endogenous Bradeion gene in a mouse genomic DNA are subjected to the suppression of expression. In this specification, a gene subjected to “suppression of expression,” or a gene “the gene expression of which has been suppressed” may mean a gene that has been genetically altered so that the biological activity of the protein encoded by this gene becomes lower than that of the same protein in its natural form. Furthermore, a gene subjected to “suppression of expression” or a gene, “the gene expression of which has been suppressed” may also mean a gene that has been genetically altered so that the protein encoded by this gene lacks the biological activity. Still further, a gene subjected to “suppression of expression” or a gene “the gene expression of which has been suppressed” may also mean a gene that has been genetically altered, so that the protein encoded by this gene is not produced. The genomic DNA wherein the expression of an endogenous Bradeion gene has been suppressed may be structurally a genomic DNA lacking the entire gene or a genomic DNA lacking a portion of the gene. Alternatively, such a genomic DNA may be a genomic DNA wherein a foreign DNA fragment has been inserted within the gene.

In the present invention, a mouse embryonic stem cell having a genomic DNA wherein the expression of a Bradeion gene has been suppressed can be produced according to the following steps.

To suppress the expression of a specific gene, a known gene targeting method is widely used. The known gene targeting method involves introducing a targeting vector for homologous recombination to take place, thereby introducing a specific mutation into a desired gene. Details about such a gene targeting method have already been described in various documents. The following steps 2 to 5 described below can be conducted according to these documents (See, e.g., Shinichi Aizawa, Gene Targeting-Production of Mutant Mice Using ES Cells, Bio Manual Series 8, YODOSHA (1995); Hogan, B., Beddington, R., Constantini, F., Lacy, E., Manipulating the Mouse Embryo, Cold Spring Harbor Laboratory Press (1994); and Joyner, A. L., Gene Targeting, A Practical Approach Series, IRL Press (1993)).

2. Production of Targeting Vector

In the present invention, a targeting vector that is used for suppressing the expression of a Bradeion gene in the genomic DNA of a mouse embryonic stem cell can be constructed, for example, as follows.

First, a region located on the 5′ side and a region located on the 3′ side of a region of a mouse Bradeion gene, into which mutation is introduced, are selected as homologous regions. DNA fragments corresponding to the regions are prepared. For this purpose, for example, plasmid clones containing mouse Bradeion genes are obtained by screening of a mouse genomic library using a mouse Bradeion cDNA. Next, a restriction enzyme map of the plasmid clones is produced, subcloning is carried out, and then the gene structure is determined. By the selection of regions that can appropriately cause homologous recombination so as to obtain desired homologous recombinants, DNA fragments homologous to the above homologous regions can be designed. At this time, it is preferable that a mouse genomic library and ES cells to be used herein are derived from the same line in order to improve recombination frequency. However, the library and ES cells may be derived from different lines. In particular, to cause the lack of the entire Bradeion gene, it is preferable to select a region upstream (5′ side) and a region downstream (3′ side) of the Bradeion gene as homologous regions. To cause the lack of a region containing one or more exons in a Bradeion gene, it is preferable to select exons, as homologous regions, located on both sides of a target region in the Bradeion gene. To insert a foreign gene into a Bradeion gene, it is preferable to select regions located on both sides of an insertion site existing in an exon, as homologous regions. DNA fragments corresponding to these regions can be respectively prepared by excising target regions using restriction enzymes from the above plasmid clones containing the mouse Bradeion genes. Alternatively, these DNA fragments may also be fragments prepared by amplifying target regions by the PCR method or may be synthesized by chemical synthesis.

Next, these DNA fragments are ligated to marker genes for selection. Generally, the above 5′-DNA fragment, a positive selection marker gene, the above 3′-DNA fragment, and a negative selection marker gene are ligated in this order, but the order of ligation is not limited thereto. Such a negative selection marker gene may not be used if it is unnecessary. DNA fragments other than the above or other compounds or the like may be optionally added. Examples of a positive selection marker gene to be incorporated into a site to which mutation is introduced include a neomycin resistance gene (Neo^{r} gene), a puromycin resistance gene, and a hygromycin B resistance gene, but are not limited thereto. Any markers can be used, as long as they are appropriately used as positive selection markers. A neomycin resistance gene is marketed as a plasmid clone (e.g., Stratagene and New England BioLabs). By inserting loxP sequences before and after such a positive selection marker gene, the marker gene, such as a neomycin resistance gene incorporated by the use of such a targeting vector into a site into which mutation is introduced, can be removed after positive selection from a genomic DNA using a restriction enzyme Cre. To linearize vectors when they are introduced into embryonic stem cells, it is preferable to previously incorporate unique restriction enzyme sites outside of homologous regions. Moreover, when homologous recombinants are screened for by Southern hybridization using probes outside the homologous regions, it is preferable to previously incorporate restriction enzyme cleavage sites for detecting the recombinant genes. These DNA fragments can be ligated according to general methods known by persons skilled in the art. Furthermore, it is convenient to ligate these DNA fragments in, for example plasmid vectors (e.g., pBluescript II SK+, Stratagene) or phage vectors.

Targeting vectors designed and/or constructed as described above can be amplified by general molecular biological techniques such as transformation of Escherichia coli and cloning by the culture thereof and then used (see e.g., J. Sambrook et al., Molecular Cloning, A Laboratory Manual, Second Edition, Cold Spring Harbor Laboratory Press (1989)).

3. Selection of Mouse Embryonic Stem Cell Wherein the Expression of Endogenous Bradeion Gene is Suppressed and Selection of Mouse Embryo into which the Cell is Introduced

To produce chimeric mice, it is required to select a preferable combination of an embryonic stem cell line and the mouse line of an embryo (in particular, an early embryo) into which the stem cell is introduced. Chimera formation rate (chimeric rate) changes depending on the combination. Hence, combinations that enable achievement of preferable chimeric rates should be selected. If there is an intention to use the produced chimeric mice for producing heterozygotes or homozygotes by crossing the chimeric mice with wild-type mice or the like, it is required that introduced embryonic stem cells contribute to the germ cell line of the chimeric mice. Accordingly, for a combination of an embryonic stem cell and the mouse line of an early embryo, which is preferably used in the present invention, it is preferably confirmed that the embryonic stem cell contributes to the germ cell line, when the embryonic stem cell (before homologous recombination) is introduced into the early embryo. Furthermore, to easily confirm the contribution rate (i.e., chimeric rate) of embryonic stem cells of a produced chimeric mouse, it is preferable to use a combination of an embryonic stem cell and the mouse line of an early embryo, with which an appropriate genetic marker can be utilized. Such a genetic marker is preferably body hair color. By the use of body hair color, a chimeric rate can be easily determined based on the appearance of mice.

Examples of a preferred combination of an embryonic stem cell line and the mouse line of an early embryo into which the embryonic stem cell line is introduced, which can be used for the chimeric mouse production of the present invention, include a combination of a 129-based embryonic stem cell line (e.g., PJ1-5 line) and a C57BL/6J mouse early embryo, and a combination of a D3-based embryonic stem cell line and C57BL/6 mouse early embryo. Embryonic stem cells and mouse strains of early embryos that can be preferably used in chimeric mouse production are also described in general experimental manuals such as “Gene Targeting” (written by A. L. Joyner, translated by Tetsuo Noda, Medical Science International; in particular, see Table 1 on page 101 and page 103) and “Experimental Medicine, separate volume, Bio Manual Series 8, Gene Targeting” (written by Shinichi Aizawa, YODOSHA (1995); in particular, see Chapter 2 IV ES Cell Culture, Table 1 on page 31 and Table 2A on page 33). However, combinations of a cell line and a mouse strain are not limited to these combinations, as long as target chimeric mice can be produced therewith.

4. Production of Mouse Embryonic Stem Cell wherein the Expression of Endogenous Bradeion Gene has been Suppressed

As a method for introducing targeting vectors prepared in “2. Production of targeting vector” into mouse embryonic stem cells, any methods known by persons skilled in the art can be used, such as a calcium phosphate transfection method, a DEAE dextran method, a lipofection method, a microinjection method, an electroporation method, and a method using a virus vector.

Of these, the electroporation method is widely used as a method for introducing targeting vectors. This method can be carried out as follows.

First, a cell suspension is used for mouse embryonic stem cells. It is prepared by culturing the strain cells on feeder cells followed by detachment from a culture plate by trypsinization. The cell suspension is preferably adjusted at a given concentration. The targeting vectors prepared as described above are linearized utilizing unique restriction enzyme cleavage sites that have been designed to be incorporated outside the homologous regions. The resultant is then mixed with the above cell suspension, and the solution is pulsed using an electroporator. After pulsing, embryonic stem cells are cultured in a culture solution for approximately 36 to 48 hours. Subsequently, an additive drug for positive selection is added to the culture solution, so that positive selection is carried out. When a positive selection marker gene used for construction of the targeting vectors is a neomycin resistance gene, G418 can be used as an additive drug for positive selection.

While exchanging everyday culture solutions containing the aforementioned additive drug, the culture of embryonic stem cells is continued. 7 to 9 days later, drug-resistant colonies that have appeared are collected.

The thus obtained drug-resistant colonies are considered to be candidate homologous recombinants. Hence, genomic DNAs extracted from these colonies are screened by Southern hybridization or PCR for embryonic stem cell lines having genomic DNAs that have experienced homologous recombination as desired.

In the above screening, Southern hybridization can be used, wherein probes that have been designed to be outside the homologous regions and used for construction of targeting vectors are utilized. Furthermore, PCR can also be used in the above screening, wherein primers that have been designed to be outside the homologous regions and used for construction of targeting vectors and primers that have been designed to be within neo genes are utilized.

5. Production of Chimeric Mouse Having Mouse Embryonic Stem Cell Introduced Therein wherein the Expression of Bradeion Gene has been Suppressed

Next, mouse embryonic stem cells wherein a desired gene mutation has been introduced by homologous recombination are introduced into mouse early embryos, thereby producing chimeric mice. As a method for producing such chimeric mice, a method known by persons skilled in the art can be used, for example, a method (blastocyst injection method) that utilizes blastocysts as mouse early embryos and involves injection of embryonic stem cells into the blastocysts and a method (aggregation method, see e.g., Andra, N et al., Proc. Natl. Acad. Sci. U.S.A., 90, 8424-8428, 1993; and Stephan. A. W. et al., Proc. Natl. Acad. Sci. U.S.A., 90, 4582-4585) that utilizes 8-cell-stage embryos or morulae as mouse early embryos and involves adhesion of such embryos to an embryonic stem cell mass.

Mouse early embryos wherein embryonic stem cells have been introduced are transferred into the uteri or oviducts of pseudopregnant mice. The embryos are generated and developed into chimeric mouse individuals and thus the chimeric mice are born. These steps are known by persons skilled in the art, and can be implemented according to various documents and experimental protocols (see, e.g., Hogan, B. et al., “Manipulating the Mouse Embryo,” Cold Spring Harbor Laboratory Press, 1988, edited by Masami Muramatsu et al.; and New Gene Engineering Handbook, 3^{rd} ed., YODOSHA (1999)).

6. Characteristics of Chimeric Mouse wherein the Expression of Bradeion Gene has been Suppressed

The chimeric mouse of the present invention produced and born as described above is a chimera wherein somatic cells and germ cells consist of original-line-derived and introduced-embryonic-stem-cell-derived cells. As a genetic marker for this chimaerism, for example, easily observable body hair color can be used. In this case, if a chimeric mouse is produced by introducing an embryonic stem cell that is derived from a mouse strain exhibiting different body hair color compared with that of an original strain from which an early embryo is derived, the rate of the contribution (chimeric rate) of the embryonic stem cell to the tissues can be calculated based on proportional comparison of the body hair color of the strain from which the embryonic stem cell is derived and that of the original strain. For example, the chimeric rate may be obtained by calculating the proportion of a specific body hair color area of the mouse strain from which the embryonic stem cell is derived to the other area; that is, based on each hair color area measured according to the appearance.

Furthermore, the chimeric mice of the present invention exhibit decreased growth in the overall cerebral nervous system, which is their unique characteristic. Furthermore, the chimeric mice of the present invention exhibit in appearance generalized decreased growth and significant malformation such as cranial dysplasia and/or visual disorders. In this specification, “generalized decreased growth” means conditions of a chimeric mouse characterized by body weight and body length that are greatly inferior to those of a normal mouse of the same age (the same week-old). Furthermore, “cranial dysplasia” means a condition of a chimeric mouse characterized by cranial bones that result in a round (hamster-like) face, compared with the long face of a normal mouse. “Cranial dysplasia” also means a condition of a chimeric mouse characterized by eyeballs that are larger with respect to its face than those of a normal mouse. Furthermore, “visual disorders” are due to decreased growth of optic nerves and this expression means a condition characterized by, in terms of appearance, wandering eyes.

As described above, because of exhibiting decreased growth in the overall cerebral nervous system and significant malformation in terms of appearance, the chimeric mice of the present invention can be used as appropriate model animals for disorders and diseases that are associated with congenital decreased growth in the cerebral nervous system and disorders and diseases that are associated with generalized decreased growth, cranial dysplasia, and visual disorders based on decreased growth of the optic nerve system. Moreover, the chimeric mice of the present invention can be used as appropriate model animals for disorders and diseases that are associated with acquired regressive conditions in the cerebral nervous system, disorders and diseases that are associated with cell canceration, and disorders and diseases that are associated with cell death, regarding which the involvement of a Bradeion protein is known. The chimeric mice of the present invention as model animals for these disorders and diseases are useful not only for elucidation of the detailed functions of a Bradeion gene, but also for elucidation of the formation and/or maintenance mechanisms of the cerebral nervous system and cellular life-span-controlling functions. Furthermore, the chimeric mice are also useful in the development of methods for treating or controlling the above disorders and diseases.

Furthermore, the fact that the chimeric mice of the present invention exhibit visual disorders based on decreased growth of optic nerves is a significant characteristic of the chimeric mice of the present invention. It has been revealed for the first time in the chimeric mice of the present invention that the suppressed expression of a Bradeion gene (reported to function site-specifically and/or cell-specifically in cerebral neurons) causes abnormalifies also in generation of optic nerves, the generation and/or differentiation of which is initiated before the same of the central nerve system. This can be a clue to reveal the molecular basis of the abnormal generation of optic nerves, which has not yet been elucidated. Moreover, this shows the possibility that the chimeric mice of the present invention can be used as model animals also for morphogenesis that takes place before the differentiation of the central nervous system in the generation stage and for disorders and diseases associated therewith.

Moreover, the chimeric mice of the present invention have germ cells wherein the expression of endogenous Bradeion genes have been suppressed, so that the chimeric mice can be used for producing heterozygotes and homozygotes by crossing them with other mice. In addition, through crossing the chimeric mice with mice having another gene mutation, gene-to-gene interaction can also be analyzed. At this time, a characteristic of exhibiting significant malformation, which is the characteristic of the chimeric mice of the present invention, can be advantageously utilized as a marker with which changes in gene functions and interactions can be easily observed. Accordingly, the chimeric mice of the present invention are useful as animals for genetic breeding.

In addition, the present invention encompasses organs, tissues, and cell populations containing cells collected from the chimeric mice of the present invention, wherein the expression of the endogenous Bradeion gene has been suppressed. These biomaterials may exhibit malformation such as hypoplasia. These biomaterials can also be used in the aforementioned analysis of gene functions and in development of methods for treating and/or controlling disorders and diseases.

### BRIEF DESCRIPTION OF THE DRAWINGS

FIG. 1 is a restriction enzyme cleavage map of subclones used for construction of targeting vectors, wherein mouse Bradeion gene fragments contained in the subclones, a Bradeion gene contained in a mouse genomic DNA, and a recombinant gene obtained by homologous recombination of the gene are shown corresponding to each other.

FIG. 2 shows the configuration of a targeting vector.

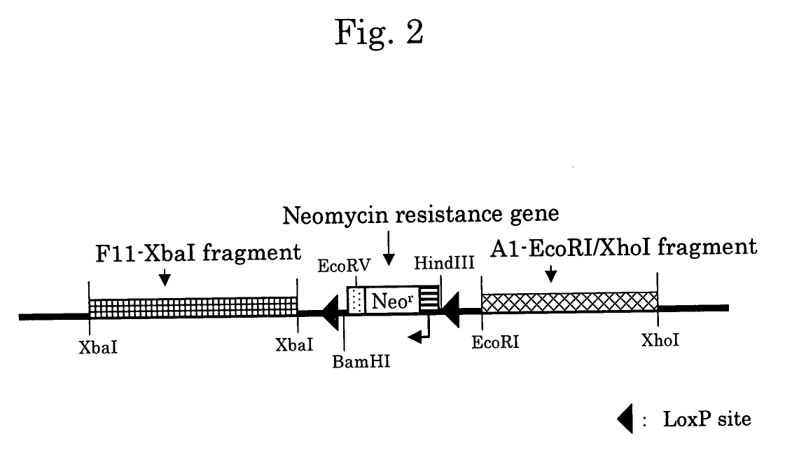

FIGS. 3A to 3D are photographs showing the malformation in appearance of chimeric mice obtained using an embryonic stem cell line 281 in the present invention. The mice in FIGS. 3A to 3D are those produced in Example 3. FIG. 3A shows a chimeric individual with identification No. 581m, FIG. 3B shows a chimeric individual with identification No. 582m, FIG. 3C shows a chimeric individual with identification No. 584f, and FIG. 3D shows a chimeric individual with identification No. 580m.

FIGS. 4A to 4D are photographs showing the malformation in appearance of chimeric mice obtained using an embryonic stem cell line 344 in the present invention. The mice in FIGS. 4A to 4D are those produced in Example 3. FIG. 4A shows a chimeric individual with identification No. 589m, FIG. 4B shows a chimeric individual with identification No. 587m, FIG. 4C shows a chimeric individual with identification No. 585f, and FIG. 4D shows a chimeric individual with identification No. 588m.

### BEST MODE FOR CARRYING OUT THE INVENTION

The present invention will be further specifically described in the following examples. However, the examples are not intended to limit the technical scope of the present invention.

### EXAMPLE 1

### Production of Targeting Vector

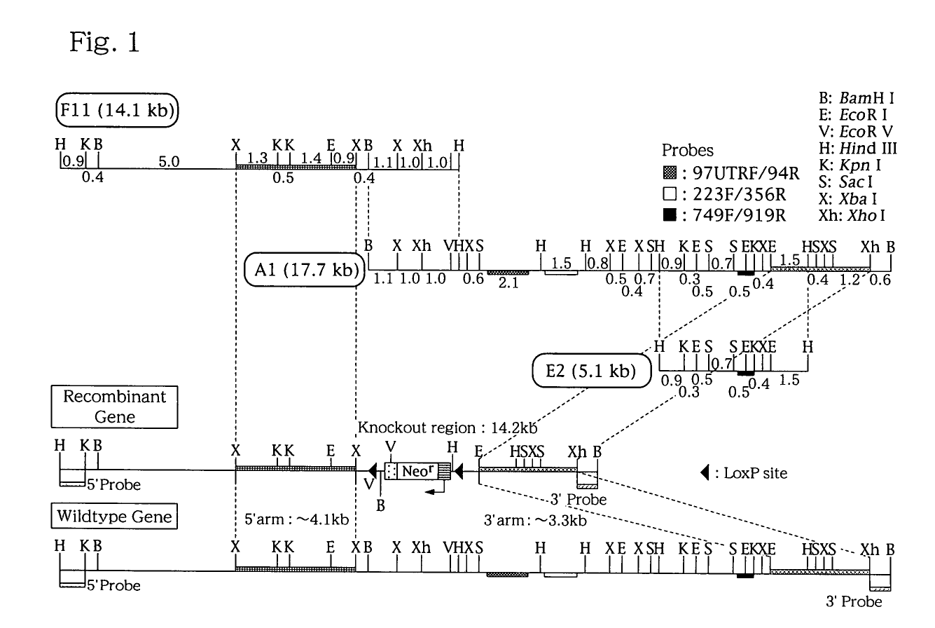

A bacterial artificial chromosome (BAC) library (produced by Incyte Genomics) of a mouse genome was screened by a conventional method using the mouse cDNA (SEQ ID NO: 1) encoding the Bradeion gene as a probe, thereby obtaining a BAC94R-C clone. The BAC clone was digested with a restriction enzyme BamH I or Hind III, and then the resultant was subcloned into a vector pZErO-1 (produced by Invitrogen). From the subclone library, plasmid clones were obtained, wherein three subclones, A1 (17.7 kb), E2 (5.1 kb), and F11 (14.1 kb) had been respectively incorporated (FIG. 1). These subclones were digested with various restriction enzymes and then the resultants were analyzed by agarose gel electrophoresis, so that a restriction enzyme map was produced (FIG. 1). As a result, it was revealed that these three types of subclones were located in order of F11, A1, and E2 from the 5′ side (FIG. 1) on the mouse genome. Next, to confirm the localization of the mouse Bradeion gene in these subclones, F11, A1, and E2 subclones were each digested with various restriction enzymes, subjected to agarose gel electrophoresis, and then subjected to Southern hybridization analysis. As probes used therefor, 3 types of DNA fragments had been prepared by the PCR method and then used. These were a sequence (97UTRF/94R) (SEQ ID NO: 2) corresponding to a 5′-untranslated region (5′ UTR), a sequence (223F/356R) (SEQ ID NO: 3) corresponding to a region within the reading frame, and a sequence (749F/919R) (SEQ ID NO: 4) corresponding to a 3′-region of the mouse Bradeion gene. In addition, the sequence (749F/919R) corresponding to the 3′-region is contained in a region corresponding to a 3′-untranslated region (3′ URF) of the mDNA sequence (SEQ ID NO: 5) of human Bradeion α in the mouse Bradeion gene. As a result of this analysis, the subclone A1 was detected by each probe, the sequence (97UTRF/94R) corresponding to the 5′-untranslated region, the sequence (223F/356R) corresponding to the region within the reading frame, and the sequence (749F/919R) corresponding to the 3′-region. Furthermore, the subclone E2 was detected by the probe of the sequence (749F/919R) corresponding to the 3′-region. The subclone F11 was not detected using any of the above 3 probes. Therefore, it was revealed that the subclone A1 contains the mouse Bradeion gene and E2 contains a portion in the latter half of the gene, but the subclone F11 does not contain the gene.

Based on the above analysis, a targeting vector was constructed to knock out the entire gene. As sequences located outside the mouse Bradeion gene and utilized for homologous recombination, a fragment (4.1 kb) excised from the subclone F11 using a restriction enzyme Xba I and a fragment (3.3 kb) excised from the subclone A1 using restriction enzymes EcoR I and Xho I were selected. These DNA fragments were excised using the restriction enzymes, and then prepared by a general method. In addition, a neomycin resistance gene to be used as a positive selection marker was prepared similarly as a fragment excised from a plasmid clone pGT-N38 (produced by New England BioLabs) using restriction enzymes Kpn I and EcoR I. Subsequently, the Xba I fragment of F11, loxP, the neomycin resistance gene, loxP, and the EcoR I/Xho I fragment of A1 were ligated in order into a vector 38 loxP, so that a targeting vector was constructed.

### EXAMPLE 2

### Production of Embryonic Stem Cell Having Allelic Gene Completely Lacking Bradeion Gene

(1) Introduction of Targeting Vector into Embryonic Stem Cell and Selection of Homologous Recombinant

An embryonic stem cell line (ES cells) PJ1-5 was subcultured and then cultured in a culture solution at 37° C. under 5% CO₂ for 36 hours. The embryonic stem cells were detached from culture plates by treatment with 3 ml of trypsin (produced by Invitrogen, 15050-065) per 100-mm culture plate. The resultants were pipetted, so that the cells were suspended in the form of single cells. 7 ml of a DMEM culture containing 10% fetal bovine serum was added to the suspension, followed by further pipetting.

Subsequently, to cause only feeder cells to be attached to the culture plates so as to separate them from the embryonic stem cells, the above cell suspension was plated on different 100-mm culture plates, and then the plates were placed in a CO₂-incubator (37° C. and 5% CO₂) for 15 to 30 minutes. Supernatants were then collected, and the supernatants collected from 2 to 5 plates were combined in a 50-ml tube, followed by centrifugation at 270 g for 5 minutes. The supernatant was aspirated, and the pellet was re-suspended in 1 ml of an ice-cooled phosphate buffer per culture plate before the detachment of the cells, so as to cause the cells to float. The number of cells in the solution was determined, and the concentration was adjusted at 7×10⁶ cells/ml.

0.8 ml of the thus obtained cell suspension (7×10⁶ cells/ml) was mixed with 40 μg of a vector DNA that had been linearized by treating the DNA with a restriction enzyme Not I. The mixture was transferred into a cuvette for electroporation (produced by BioRad, Cat. No. 165-2088). The mixture in the cuvette was pulsed at 500 μF and at 240V using an electroporator (produced by BioRad, Genepulser). Next, the cuvette was removed from a cuvette holder, and then allowed to stand on ice for 20 minutes. Subsequently, the cell suspension in the cuvette was transferred into 10 to 20 ml of the above culture solution containing mouse leukemia inhibiting factor (LIF).

Next, the cell suspension suspended in the culture solution containing LIF was plated at 10 ml per culture plate coated with gelatine. On the next day, culture solutions were exchanged (culture solutions containing LIF were used). 2 days later, culture was carried out after adding 150 to 250 μg/ml G418 (drug for selection) to the culture solutions containing LIF. Observation of colonies was continued while exchanging culture solutions every day. A plurality of drug-resistant colonies that appeared approximately 8 days after selection were collected. Each colony was cloned and the lines thereof were established.

(2) Identification of Homologous Recombinant by Southern Hybridization

The above-obtained drug-resistant colonies (which appeared approximately 8 days after selection using G418) were screened by Southern hybridization analysis. Homologous recombinants lacking the entire Bradeion gene were identified.

For this purpose, first, genomic DNAs were extracted according to a general method from culture cells (for genomic DNA extraction) of each of embryonic stem cell lines 279, 281, 313, and 344 derived from the drug-resistant colonies that were candidate homologous recombinants lacking the entire Bradeion gene. Each genomic DNA was digested with a restriction enzyme BamH I or Hind III, thereby preparing samples. Both samples were subjected to agarose gel electrophoresis, and then the resultants were blotted onto nylon membranes.

Southern hybridization was carried out 3 times for every probe by the following procedures. First, prehybridization was carried out at 65° C. for 30 minutes using a prehybridization solution. Subsequently, hybridization was carried out at 65° C. overnight using a hybridization solution and separately using the following probes (with 1 type of probe used at a time). Next, each resultant was washed at 65° C. for 15 minutes using a 0.1×SSC-0.1×SDS solution, followed by similar washing. The membranes were subjected to signal analyses using an image analyzer (BAS 2000).

As probes, a 5′ probe recognizing a genomic sequence upstream of the Bradeion gene, a Neo probe recognizing a neomycin resistance gene, and a 3′ probe recognizing a genomic sequence downstream of the Bradeion gene were separately used. The 5′ probe is a 0.9-kb DNA fragment prepared as a fragment excised with Kpn I and Hind III from the subclone F11 of Example 1. The 5′ probe recognizes a sequence upstream of the Bradeion gene as shown in FIG. 1 in the gene that experienced homologous recombination or a wild-type gene. The 3′ probe is a 0.6-kb DNA fragment prepared as a fragment excised with BamH I and Xho I from the subclone A1 of Example 1. The 3′ probe recognizes a sequence immediately downstream of the Bradeion gene as shown in FIG. 1 in the gene that experienced homologous recombination or the wild-type gene. The Neo probe is a 1.8-kb DNA fragment prepared as a fragment excised with BamH I and EcoR I from a Final vector (produced by Incyte Genomics). As shown in FIG. 1, the Neo probe recognizes the neomycine resistance gene (Neo) that has been incorporated into the genome by homologous recombination with the targeting vector. These probes were labeled with alpha-CTP³² by using a Rediprime II DN Labelling System (Amersham Phramacia Biotech) and then used.

As a result of analysis using these probes, in the case of the samples digested with BamH I, a 5.7-kilobase band signal was recognized in the DNA sample derived from the embryonic stem cells that had experienced homologous recombination and a 17.7-kilobase band signal was recognized in the DNA sample containing only the wild-type allelic gene derived from the embryonic stem cells that had not experienced homologous recombination. In the case of the samples digested in Hind III, a 12.2-kilobase band signal was detected in the DNA sample derived from the embryonic stem cells that had experienced homologous recombination and a 14.1-kilobase band signal was detected in the DNA sample containing only the wild-type allelic gene derived from the embryonic stem cells that had not experienced homologous recombination.

As a result of such Southern hybridization, lines 281 and 344 were identified as embryonic stem cell lines having genomic DNAs wherein the expression of the endogenous Bradeion gene had been suppressed. Therefore, to inject the embryonic stem cells into blastocysts, the embryonic stem cell lines 281 and 344 were selected.

### EXAMPLE 3

### Production of Chimeric Mouse Using Mouse Embryonic Stem Cell having Genome Lacking Bradeion Gene and Morphological Observation of the Chimeric Mouse

The embryonic stem cell lines 281 and 344 were each injected into blastocysts of C57BL/6 mice by microinjection. According to a conventional method, the blastocysts were then transferred into the oviducts of pseudopregnant female mice for the embryos to generate and develop into mice.

As a result, the following chimeric mice were obtained.

(1) Chimeric Mice Obtained Using the Embryonic Stem Cell Line 281 (Born on May 6, 2001)

Chimeric individual (identification No. 580m) . . . chimeric rate: 90%, male
Chimeric individual (identification No. 581m) . . . chimeric rate: 90%, male
Chimeric individual (identification No. 582m) . . . chimeric rate: 90%, male
Chimeric individual (identification No. 583m) . . . chimeric rate: 90%, male
Chimeric individual (identification No. 584f) . . . chimeric rate: 95%, female
   (2) Chimeric Mice Obtained Using the Embryonic Stem Cell Line 344 (Born on May 8, 2001)
Chimeric individual (identification No. 585f) . . . chimeric rate: 90%, female
Chimeric individual (identification No. 587m) . . . chimeric rate: 98%, male
Chimeric individual (identification No. 588m) . . . chimeric rate: 98%, male
Chimeric individual (identification No. 589m) . . . chimeric rate: 90%, male

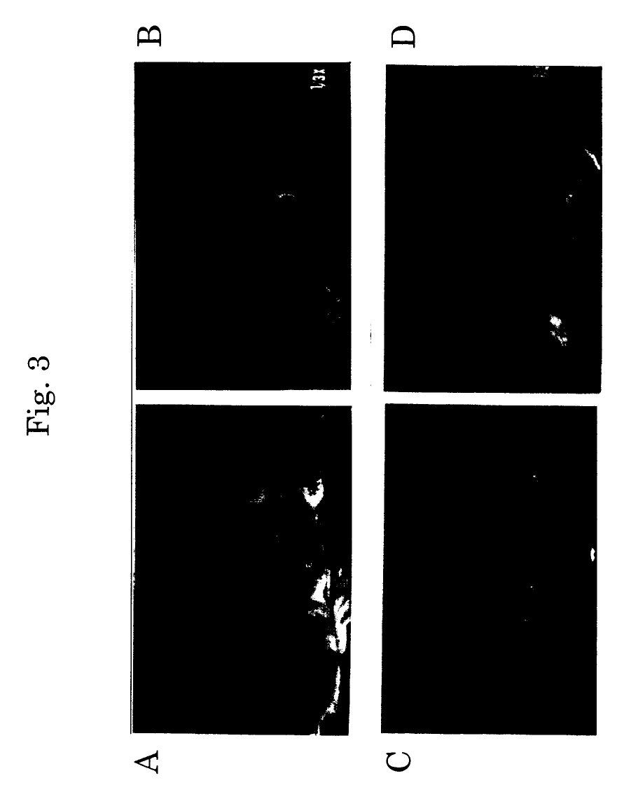

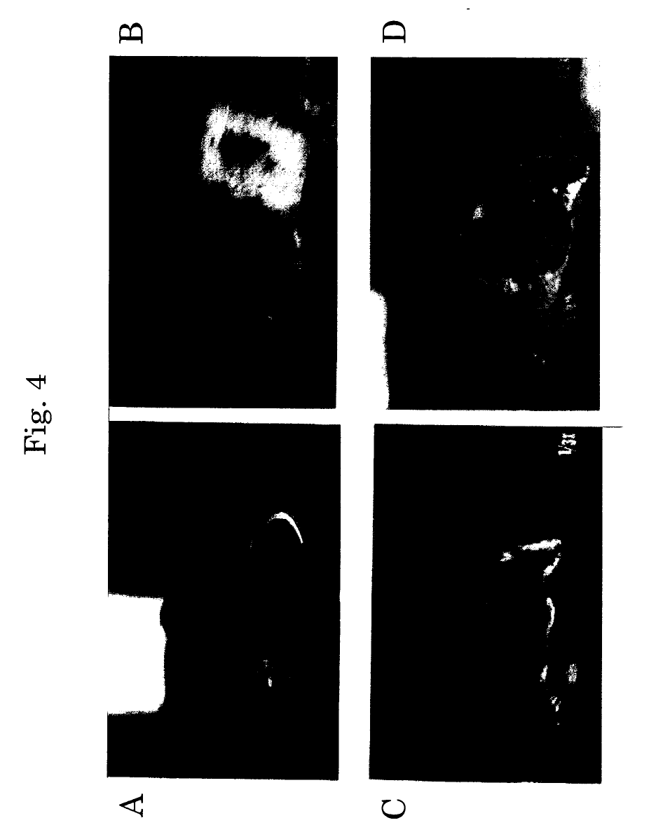

FIGS. 3 and 4 are photographs showing the appearance of the chimeric mice obtained above. The photographs of FIG. 3 show chimeric mice obtained using the embryonic stem cell line 281. The photographs of FIG. 4 show the chimeric mice obtained using the embryonic stem cell line 344. In all the chimeric mice, generalized decreased growth, hamster-like faces, relatively large eyeballs, and wandering eyes were observed.

All publications, patents and patent applications cited herein are incorporated herein by reference in their entirety.

### INDUSTRIAL APPLICABILITY

The present invention provides chimeric mice exhibiting hypoplasia in the cerebral nervous system and various types of malformation and having an endogenous Bradeion gene, the gene expression of which has been suppressed from the time of generation. The chimeric mice can be usefully utilized as model animals and animals for genetic breeding associated with abnormalities in the cerebral nervous system.

1. A chimeric mouse generated from a mouse embryo, wherein the mouse embryo has a mouse embryonic stem cell introduced therein that has a genomic DNA containing an endogenous Bradeion gene, the gene expression of which has been suppressed.

2. The chimeric mouse of claim 1, wherein the endogenous Bradeion gene, the gene expression of which has been suppressed, has been genetically altered so as to encode a Bradeion protein lacking biological activity.

3. The chimeric mouse of claim 1, wherein the endogenous Bradeion gene, the gene expression of which has been suppressed, has been genetically altered so as to lack the entire coding region.

4. The chimeric mouse of any one of claims 1 to 3, wherein the mouse embryonic stem cell is derived from a PJ1-5 line and the mouse embryo is derived from a C57BL/6 mouse.

5. The chimeric mouse of any one of claims 1 to 4, wherein the mouse embryo is selected from the group consisting of 8-cell-stage embryos, morulae, and blastocysts.

6. The chimeric mouse of any one of claims 1 to 5, which exhibits malformation.

7. The chimeric mouse of claim 6, wherein the malformation is at least one selected from the group consisting of cranial dysplasia, visual disorders, and generalized decreased growth.

8. The chimeric mouse of any one of claims 1 to 7, wherein the chimeric rate is 90% or more and less than 98%.

9. A cell derived from the mouse embryonic stem cell, which is collected from the chimeric mouse of any one of claims 1 to 8.
